(19) United States
(12) Patent Application Publication
(10) Pub. No.: US 2021/0368816 A1
(43) Pub. Date: Dec. 2, 2021

(54) Native whey protein for reducing allergy

(71) Applicant: N.V. Nutricia, Zoetermeer (NL)

(72) Inventors: Betty Lobato-Van Esch, Utrecht (NL); Gerrit Hols, Utrecht (NL)

(73) Assignee: N.V. Nutricia, Zoetermeer (NL)

(21) Appl. No.: 16/968,167

(22) PCT Filed: Feb. 15, 2019
(86) PCT No.: PCT/NL2019/050102

§ 371 Date: Aug. 7, 2020

(30) Foreign Application Priority Data
Feb. 15, 2018 (NL) PCT/NL2018/050103
Nov. 14, 2018 (NL) PCT/NL2018/050763

(51) Int. Cl. A23C 9/142; A23L 33/19; A23L 33/00; A23C 9/15; A23C 3/02; A23C 9/16; A61K 39/35; A61P 37/08

(52) U.S. Cl. CPC A23C 9/1422; A23L 33/19; A23L 33/40; A23C 9/1512; A23C 3/02; A23C 2210/208; A61K 39/35; A61P 37/08; A23V 2002/00; A23C 2210/206; A23C 9/16

(57) ABSTRACT

The invention concerns an infant formula product comprising intact and native whey protein for use in reducing or preventing allergic response. The infant formula product comprises whey protein having a nativity value of at least 90% and/or which is obtainable by a process comprising: (a) processing defatted milk into a casein stream, a whey protein stream and a lactose stream, by: (i) subjecting the defatted milk to microfiltration over a membrane capable of retaining bacteria and permeating milk proteins or to a pasteurization step, to provide a debacterialized milk; (ii) subjecting the permeate originating from step (i) to microfiltration over a membrane capable of retaining casein and permeating whey proteins, to provide a casein stream as retentate and a permeate comprising whey protein; (iii) fractionating the permeate originating from step (ii) into a whey protein stream and a lactose stream; (b) combining at least part of the casein stream, at least part of the whey protein stream originating from step (a) and a lactose source to obtain a recombined stream; (c) optionally pasteurization of the recombined stream from step (b), (d) using the recombined stream originating from step (b) or (c) in the manufacture of the infant formula product.

### FIELD OF THE INVENTION

The present invention relates to the field of infant formula products, in particular for reducing and/or preventing allergic response.

### BACKGROUND

One approach towards reducing the occurrence of allergic response, is the avoidance of ingestion of the food product, typically protein, in question, to which the subject is allergic. This approach requires for strict control of the diet of the subject, often combined with supplementation of deficient nutrients, caused by the lack of ingestion of one particular food product. Furthermore, cross-contamination in food production may still cause allergic responses to contaminants which are not expected to be present in a particular food product. For some vulnerable subject groups, such strict dietary control is not or only very limited possible, such as for example in infants who require a carefully balanced diet especially in the first months of life, in which milk proteins play a crucial role. Hence, an alternative approach, which is often used in infant formulae, is to avoid the use of intact milk proteins but to resort to the use of hydrolysed protein, wherein the epitopes in the protein that cause the allergic response are destroyed prior to ingestion.

There is literature available wherein the protective effect of unprocessed farm milk consumption on childhood asthma and atopy has been studied using an epidemiological angle. Asthma, atopy, and hay fever were associated to reported milk consumption and for the first time to objectively measured milk constituents by using regression analyses. Despite the extent of the study wherein 8334 subjects were followed, the outcome was largely inconclusive on the mechanism of action and the role of particular constituents of farm milk. Also, whereas it was reported that the protective effect of raw milk consumption on asthma might be associated with the whey protein fraction of milk, the outcome of the study does not provide clues as to which components are responsible for the studied farm milk effects (J. Allergy Clin. Immunol. 2011; 128:766-73).

WO2013/011040 (Ludwig-Maximilians-Universitat, Austria) discloses a dehydrated raw milk preparation that is expressly mentioned not to be heat-treated but obtained via freeze-drying. It also discloses that raw milk is rich in microorganisms and that it is conceivable that the microorganisms stimulate expression of innate immunity receptor genes. It concludes that it remains an open question whether increased expression of Toll-Like Receptors (TLRs) associated with raw milk consumption reflects a relevant pathway underlying allergic diseases development or whether it is merely an indicator of exposure to microbes. It also concludes that the study underlying the claims of WO2013/011040 does not allow to answer whether the up-regulation of innate immune receptors directly modulates the development of allergic disease or whether it is a marker for the effect of genes and environment on allergic disease.

Moreover, native proteins are not readily incorporated into infant formula. Current food safety regulations govern that milk containing food products receive a heat treatment that is sufficiently high to deactivate certain enzymes present in the raw milk. For example, European Regulation 2074/05 requires that infant formulae receive a heat treatment sufficient to deactivate the enzymes in order that an alkaline phosphatase test produces a negative result. This is reflected in the nativity values of a range of infant formulae, tested herein, which all contain substantial amounts of deactivated whey protein.

WO2013/068653 (Valio LTD, Finland) discloses a method for the production of an infant formula base relying on a series of filtration steps to keep protein nativity levels high. Also, WO2013/068653 teaches that whey protein nativity is retained for more than 90% when skim milk is subjected to a pasteurization step. And WO2013/068653 teaches to hydrolyze proteins to enable a hypoallergenic infant formula base to be produced.

The present invention provides in the need in the art for an intact, native whey protein fraction to be used for reducing the severity or magnitude of allergic response.

### SUMMARY OF THE INVENTION

The inventors surprisingly found that the native, intact whey protein according to the invention significantly reduced the occurrence of allergic response, typically allergic skin response. The native, intact whey protein is comprised in a nutritional composition, preferably an infant formula product. The present invention concerns the use of the native, intact whey protein according to the invention for reducing and/or preventing allergic response and an infant formula product comprising the native whey protein according to the invention.

In a first aspect, the invention concerns an infant formula product comprising intact whey protein, wherein the infant formula product is obtainable by a process comprising:
(a) processing defatted milk into a casein stream, a whey protein stream and a lactose stream, by:
   (i) subjecting the defatted milk to microfiltration over a membrane capable of retaining bacteria and permeating milk proteins or to a pasteurization step, to provide a debacterialized milk;
   (ii) subjecting the permeate originating from step (i) to microfiltration over a membrane capable of retaining casein and permeating whey proteins, to provide a casein stream as retentate and a permeate comprising whey protein;
   (iii) fractionating the permeate originating from step (ii) into a whey protein stream and a lactose stream;
(b) combining at least part of the casein stream, at least part of the whey protein stream originating from step (a) and a lactose source to obtain a recombined stream;
(c) optionally pasteurization of the recombined stream from step (b),
(d) using the recombined stream originating from step (b) or (c) in the manufacture of the infant formula product, for use in reducing and/or preventing allergic response.

Alternatively, the invention concerns an infant formula product comprising whey protein, wherein the whey protein is intact and native for use in reducing and/or preventing allergic response.

The infant formula product of the invention contains native whey protein. In a preferred embodiment, the whey proteins have a nativity value of more than 92%, preferably more than 94%, more than 95% or even more than 98%. Preferably, substantially no non-native whey protein is comprised in the infant formula product according to the invention.

The infant formula product of the present invention contains intact whey proteins. Intact means that the whey proteins have not been subjected to a hydrolysis step. Thus, substantially no non-intact whey protein is comprised in the infant formula product according to the invention.

In a preferred embodiment, the infant formula is pasteurized. The inventors of the present invention have surprisingly shown that an infant formula product with intact whey protein, obtained according to the present invention that includes a pasteurization step, can be used for reducing and/or prevention of an allergic response. Alternatively worded the infant formula is substantially free of alkaline phosphatase activity. In a preferred embodiment, the infant formula is a liquid, ready-to-feed infant formula which is substantially free of alkaline phosphatase activity. In a preferred embodiment, the term substantially free of alkaline phosphatase activity means that, when measured using a liquid, ready-to-feed infant formula, the alkaline phosphatase activity is below 350 mU/L.

Still, in another preferred embodiment, the infant formula product is not pasteurized. The inventors of the present invention have surprisingly shown that an infant formula product with intact whey protein, obtained according to the present invention without the inclusion of a pasteurization step, can be used for reducing and/or prevention of an allergic response. Alternatively worded the infant formula contains alkaline phosphatase activity. In this preferred embodiment, the infant formula product is a liquid, ready-to-feed product and contains alkaline phosphatase activity or is considered alkaline phosphatase positive. The alkaline phosphatase activity of the infant formula product in this embodiment is above 350 mU/L.

In a preferred embodiment, the invention relates to an infant formula product comprising whey protein, wherein the whey protein is intact and native, as defined by a nativity value of at least 90%, for use in reducing and/or preventing allergic response. The infant formula product is preferably pasteurized and substantially free of alkaline phosphatase activity. Alternatively, the infant formula product is not pasteurized and contains alkaline phosphatase activity.

### DETAILED DESCRIPTION

The inventors surprisingly found that the native whey protein according to the invention significantly reduced the occurrence of allergic response. The native whey protein is comprised in a nutritional composition, preferably an infant formula product.

The inventors have developed a process for preparing infant formulae products, containing native whey protein. In the context of the present invention, infant formula can also be referred to as a synthetic formula. Human milk is not considered to be an infant formula.

### Composition

The composition according to the invention is a nutritional composition, preferably an infant formula product. In the context of the present invention, “infant formula product” refers to milk-based nutritional compositions suitable for feeding infants, which typically are in the form of a reconstitutable powder or a ready-to-feed liquid composition, or refers to infant formula bases, which are suitable for making infant formulae and which comprise all or almost all essential ingredients in the required amounts for infant nutrition. Preferably, the composition is an infant formula, a follow-on formula, a growing-up milk, or a base therefore. Most preferably, the composition is an infant formula. The infant formula product may be a powder, preferably a spray-dried powder, intended to be reconstituted into a liquid infant formula, or a liquid infant formula.

The composition according to the invention can be defined in two distinct ways. In one embodiment, the composition according to the invention is defined by the process of preparing the composition. In one embodiment, the composition is defined by the presence of a whey protein which is intact and native. In an especially preferred embodiment, the composition according to the invention is defined by the process of preparing the composition and by the presence of a whey protein which is intact and native.

In one embodiment, the composition according to the invention comprises a whey protein fraction obtainable by the process according to the invention as defined below, in particular step (a) and optionally step (c). Thus, in one embodiment, the composition according to the present invention comprises a whey protein fraction which is obtainable as a whey protein stream via the process of the present invention as defined below, in particular step (a). In particular, the whey proteins are obtainable by subjecting a defatted, debacterialized milk to microfiltration over a membrane capable of retaining casein and permeating whey proteins to provide a permeate comprising whey protein and fractionating the permeate into a whey protein stream and a lactose stream, wherein debacterization is preferably performed by microfiltration or pasteurization. The whey proteins are present in the thus obtained ultrafiltration retentate. In one embodiment, the whey protein is obtained by the process defined herein. It is well-known to the skilled person how to obtain such an ultrafiltration retentate that contains native whey proteins starting from defatted milk.

In one embodiment, the composition according to the invention is defined by the process of preparing the composition. This process is herein referred to as the process according to the invention. The process according to the invention comprises:
(a) processing defatted milk into a casein stream, a whey protein stream and a lactose stream, by:
   (i) subjecting the defatted milk to microfiltration over a membrane capable of retaining bacteria and permeating milk proteins or to a pasteurization step, to provide a debacterialized milk;
   (ii) subjecting the permeate originating from step (i) to microfiltration over a membrane capable of retaining casein and permeating whey proteins, to provide a casein stream as retentate and a permeate comprising whey protein;
   (iii) fractionating the permeate originating from step (ii) into a whey protein stream and a lactose stream;
(b) combining at least part of the casein stream, at least part of the whey protein stream originating from step (a) and a lactose source to obtain a recombined stream;
(c) optionally pasteurization of the recombined stream from step (b),
(d) using the recombined stream originating from step (b) or (c) in the manufacture of the infant formula product.

In the process according to the invention defatted milk is treated to produce an infant formula product. In the context of the present invention, whenever a certain stream or composition is mentioned to “originate from” a certain process step, such as from the recombined stream originating from step (b), said stream or composition can be the composition which is directly obtained by said process step. In addition, if such a directly obtained stream or composition undergoes one or more additional processing steps, such as partial evaporation and/or supplementation of additional water or other components, the stream or composition is also regarded to originate from that specific process step. Thus, if the recombined stream of step (b) would be partially evaporated prior to it is entered in the pasteurization step (c), the incoming stream of step (c) is still regarded to be the recombined stream originating from step (b). In the context of the present invention, the term “stream” refers to a liquid composition, although the presence of some solid material is not excluded, e.g. as in a suspension, as long as the composition can be handled by conventional dairy plants.

The present process uses milk as starting material in step (a). Defatted milk, preferably defatted cow's milk, is subjected to step (a). In the context of the invention, “defatted milk” refers to milk having a reduced fat content compared to whole milk. Typically, the fat content of the defatted milk is in the range of 0-2 wt %, preferably 0-1 wt %, more preferably 0-0.2 wt %, most preferably 0-0.05 wt %, based on total weight of the defatted milk. In one embodiment, the defatted milk is skim milk. The present process employs milk, which refers to non-human milk, preferably cow's milk. Most preferably, cow's skim milk is used. In one embodiment, the process comprises a step of defatting milk to obtain the defatted milk, which is subsequently subjected to step (a). Herein, non-defatted milk, or just milk or whole milk, is subjected to the defatting step. The defatting step affords the defatted milk. Preferably, the defatted milk is the sole protein source for the infant formula product.

### Step (a)

In step (a), the defatted milk is processed or fractioned into a casein stream, a whey protein stream and a lactose stream. Herein, the casein stream is a liquid composition comprising casein, which is enriched in casein compared to the casein content in the incoming defatted milk, the whey protein stream is a liquid composition comprising whey protein, which is enriched in whey protein compared to the whey protein content in the incoming defatted milk and the lactose stream is a liquid composition comprising lactose, which is enriched in lactose compared to the lactose content in the incoming defatted milk. In the context of the present invention, “enriched” is defined that the content of the enriched component, based on dry weight, is increased in one stream compared to another stream. Thus, the casein stream is enriched in casein, i.e. has a higher casein content, based on dry matter, compared to the incoming defatted milk.

The fractionation of step (a) is accomplished by membrane filtration techniques and involves a combination of microfiltration and ultrafiltration. The casein stream originates from the microfiltration as retentate, the whey protein stream originates from the ultrafiltration as retentate and the lactose stream originates from the ultrafiltration as permeate. Suitable membrane filtration processes are known in the art, e.g. as disclosed in WO 2013/068653, WO 2013/137714 and WO 2015/041529. More specifically, step (a) includes:
(i) subjecting the defatted milk to microfiltration over a membrane capable of retaining bacteria and permeating milk proteins or to a pasteurization step, to provide a debacterialized milk;
(ii) subjecting the permeate originating from step (i) to microfiltration over a membrane capable of retaining casein and permeating whey proteins, to provide a casein stream as retentate and a permeate comprising whey protein; and
(iii) fractionating the permeate originating from step (ii) into a whey protein stream and a lactose stream.

The incoming defatted milk is subjected to debacterization (bacterial removal) in step (i). Debacterization may be performed by filtration or by pasteurization. In one embodiment, debacterization is performed by bacterial filtration (e.g. microfiltration (MF)). Such filtration processes to reduce the bacterial load of milk are known in the art. The microfiltration of step (i) may be performed by microfiltration over a membrane capable of retaining bacteria and permeating milk proteins, to provide a debacterialized milk as permeate. Preferably, the microfiltration of step (i) comprises ceramic microfiltration. The MF membrane preferably has a pore size of between 1.8 and 0.6 μm, preferably between 1.4 and 0.8 μm. The MF process of step (i) is preferably executed at a temperature of between 4 and 20° C., more preferably between 8 and 15° C., most preferably at a temperature of about 10° C.

Alternatively, step (i) is performed by pasteurization. Pasteurization of defatted milk in order to reduce the bacterial load of the milk is well-known in the art. Pasteurization and preferred embodiments thereof are described in more detail below in the context of step (c), which equally applies here.

In the microfiltration step (ii), the debacterialized milk originating from step (i) is fractioned into two distinct streams, each enriched in a particular protein type; a casein enriched MF retentate (MFR) and a whey protein enriched MF permeate (MFP) are produced. The MF step (ii) is performed over a membrane that enables fractionation of casein and whey proteins. Such a membrane typically has a porosity of between 0.05 and 0.5 μm, more preferably between 0.08-0.35 μm. Alternatively, the membrane used in step (ii) may have a molecular weight cut-off in the range of 250-1500 kDa, preferably in the range of 500-1000 kDa. Preferably, a ceramic membrane or a spiral wound (organic) membrane is used. Microfiltration of step (ii) is preferably performed with a volume concentration factor (VCF) in the range of 1.5-10, preferably 2-5, which has been found to provide the most optimal results in terms of the composition of the MF retentate, especially in terms in terms of casein content.

In the context of the invention, the term “volume concentration factor” or “VCF” is the factor at which a liquid composition is concentrated upon filtration, i.e. the total volume of the incoming stream prior to filtration divided by the total volume of the retentate after filtration, irrespective of the total solid content. Thus, when 5 L of a liquid composition is fractionated over an ultrafiltration membrane into a permeate of 4 L and a retentate of 1 L, this UF process operates with a VCF of 5/1=5.

According to a preferred embodiment, microfiltration of step (ii) is enhanced with diafiltration (DF). Diafiltration may be accomplished by diluting the retentate of the MF at least once with an amount of water, or by diluting the incoming debacterialized milk with an amount of water and subjecting the diluted milk to MF. The DF water may be added to the incoming debacterialized milk or MFR at once, or the total amount of DF water may be added in several fractions. After each addition of DF water to the incoming skim milk or MFR, the diluted liquid composition is subjected to MF.

Fractionation of a composition comprising whey protein and lactose into a composition enriched in whey protein and a composition enriched in lactose is known in the art. Step (iii) is preferably performed by ultrafiltration (UF). During ultrafiltration, most of the liquid and small solutes end up in the UF permeate (UFP), while the UF retentate (UFR) comprises substantially all whey protein, in a smaller volume. Small molecules which permeate through the UF membrane are for example lactose, monovalent and polyvalent ions. The ultrafiltration of step (iii) can be carried out with any UF membrane known in the art, including ceramic membranes, tubular and organic spiral wound membranes. Preferably the UF membrane is an organic spiral wound membrane. The UF membrane has a molecular weight cut-off of that enables proteins, preferably whey proteins, to remain in the retentate, and allow small solutes, for example lactose, to permeate through the membrane. The UF step (iii) preferably is carried out with a membrane having a molecular weight cut-off of at most 25 kDa, more preferably at most 10 kDa, and preferably of at least 2.5 kDa, more preferably at least 5 kDa. The UF step (iii) is preferably carried out with a volume concentration factor (VCF) in the range of 20-200, preferably 50-150, which has been found to provide the most optimal results in terms of the composition of the UF retentate.

Step (a) may further comprise one or more concentration steps, such as concentration of the MFR originating form step (ii) and/or the UFR originating form step (iii). Concentration is preferably performed by reverse osmosis (RO), nanofiltration (NF) and/or evaporation. NF is most preferred, as NF concentrates the stream and at the same time lowers the monovalent ion content, which are able to permeate the NF membrane. Such lowering of the monovalent ion content is typically desirable in the production of infant formula products.

The protein fraction of the casein stream originating from step (a) typically comprises very little whey protein, preferably less than 15 wt %, more preferably less than 10 wt %, based on the weight of the protein fraction of the casein stream, and is high in casein. Preferably the protein fraction comprises at least 85 wt % casein, more preferably at least 90 wt % casein. The content of total solids in the casein stream typically ranges from 5 to 30 wt %, preferably from 7 to 30 wt %, most preferably from 17 to 24 wt %, based on total weight of the casein stream. The casein stream may also be referred to as a casein concentrate, casein isolate, micellar casein concentrate or micellar casein isolate (MCI).

The whey protein stream is typically a liquid composition having a total solid content of 5-35 wt %, preferably of 10-30 wt %, most preferably of 20-30 wt %, and typically comprises 25-90 wt %, preferably 60-85 wt % whey proteins based on total dry weight. The whey protein stream may also be referred to as an aqueous composition comprising whey proteins. Although the whey protein stream is enriched in whey protein compared to the incoming defatted milk, it may still contain substantial amounts of casein, depending on the exact conditions at which the fractionation between casein and whey protein by ultrafiltration, is performed. In one embodiment, the whey protein stream comprises at most 40 wt %, preferably 5-20 wt % casein, based on total weight of the protein. Such variations in the fractionation conditions and the accompanying changes in the whey protein stream are known in the art. Depending on the amount of casein present in the whey protein stream, the amount of casein used in combining step (b) can be adapted such that the infant formula product has a whey protein:casein ratio that falls within the preferred ratio of 90:10 to 40:60.

The lactose stream is typically a liquid composition having a total solid content of 3-30 wt %, preferably of 5-22 wt %. The lactose content in the lactose stream originating from step (a) is typically at least 75 wt %, preferably at least 90 wt % or even at least 95 wt %, based on total dry weight.

### Demineralization

The process according to the invention preferably comprises a demineralization step, wherein the lactose source, or one or more components thereof, is/are demineralized prior to being subjected to step (b). Demineralization is thus typically performed on at least part of the lactose stream originating from step (a) prior to being subjected to step (b). Demineralization is particularly preferred for the manufacture of infant formula products, for which it is typically required to lower the mineral content as compared to the incoming milk. Thus, in one embodiment, at least part of the lactose stream originating from step (a), preferably the UFP originating from step (iii), is subjected to demineralization prior to being used as (part of) the lactose source in step (b).

Demineralization of the lactose source may be performed by any technique known in the art, such as electrodialysis, ion exchange, salt precipitation, lactose crystallization, membrane filtration techniques such as nanofiltration, optionally enhanced with diafiltration, or combinations thereof. In a preferred embodiment, demineralization comprises at least one of salt precipitation, electrodialysis, lactose crystallization and ion exchange, optionally in combination with nanofiltration, more preferably demineralization comprises nanofiltration in combination with at least one of salt precipitation, electrodialysis, lactose crystallization and ion exchange. In preferred embodiment, demineralization comprises at least electrodialysis and/or salt precipitation. In one preferred embodiment, demineralization comprises at least nanofiltration in combination with electrodialysis and/or salt precipitation. The inventors found that when only nanofiltration is used for demineralization, especially for demineralization of an ultrafiltration permeate as lactose source in the preparation of infant formula products, the content of divalent ions, such as calcium and phosphate, is typically insufficiently reduced to obtain a final infant formula product within legal requirement.

Demineralization is preferably performed such that at least 20 wt %, or preferably 50 wt %, more preferably at least 70 wt % or at least 80 wt %, most preferably at least 90 wt % of the polyvalent ions and/or such that at least 20 wt % of the monovalent ions are removed, more preferably at least 35 wt % or at least 50 wt %, most preferably at least 60 wt % of the monovalent ions, present in the lactose stream, e.g.t the UFP originating from step (iii), are removed.

### Step (b)

In step (b), at least part of the casein stream, at least part of the whey protein stream originating from step (a) and a lactose source are combined to obtain a recombined stream. This recombined stream is used to manufacture the infant formula product in step (d), optionally after a pasteurization step (c). The combining of step (b) affords a composition having a protein fraction comprising both casein and whey protein in a certain weight ratio. The combining of step (b) may involve additional components. The combining is preferably done such that the whey protein to casein weight ratio in the recombined stream is in the range of 90:10 to 40:60, more preferably in the range of 80:20 to 50:50, even more preferably in the range of 75:25 to 50:50, most preferably in the range of 70:30 to 55:45. In one embodiment, the whey protein to casein weight ratio in the recombined stream is about 60:40. The exact ratio is typically determined by the type of infant formula product that is being produced, and can be adjusted as known in the art. In addition, much attention in the art is given to the amino acid profile of infant formula products. The process according to the invention provides optimal flexibility in targeting a specific desired amino acid profile, e.g. by adjusting the ratio in which the whey protein and casein streams are combined or in varying the specific process conditions of the microfiltration of step (a). As such, optimal amino acid profiles resembling those found in human milk are obtainable with the process according to the invention.

In one embodiment, 10-50 wt %, preferably 12-25 wt %, based on total weight of the casein, of the casein stream originating from step (a) is subjected to step (b). Most preferably, about 16 wt %, based on total weight of the casein, of the casein stream originating from step (a) is subjected to step (b). The amount of the casein stream originating from step (a) that is subjected to step (b) is advantageously governed by the desired whey protein to casein weight ratio in the recombined stream. Preferably, all of the whey protein stream originating from step (a) is subjected to the combining of step (b). In one embodiment, 0-50 wt %, preferably 5-25 wt %, based on total weight of the lactose, of the lactose stream originating from step (a) is subjected to step (b) as (part of) the lactose source. The amount of the lactose stream originating from step (a) that is subjected to step (b) as (part of) the lactose source is advantageously governed by the amount of lactose required for step (d). In case the amount of lactose in the lactose stream originating from step (a) that is subjected to step (b) would be insufficient for infant formula product manufacture, additional lactose can be used. In one embodiment, part of the casein stream is combined with all of the whey protein stream and part of the lactose stream. In one embodiment, part of the casein stream is combined with all of the whey protein stream and all of the lactose stream. In one embodiment, part of the casein stream is combined with all of the whey protein stream and nothing of the lactose stream. In one embodiment, part of the MFR originating from step (ii) is combined with at least part of the UFR originating from step (iii) and at least part of the UFP originating from step (iii).

In step (b), three or more streams are recombined into one stream. This recombining may occur at once (streams are combined simultaneously) or step-wise (streams are combined consecutively). Combining can be performed as wet mixing or as dry mixing or even as a combination of both. Preferably, the combining occurs as wet mixing, wherein liquid compositions are mixed in the appropriate amounts.

### Step (c)

The process according to the invention may contain a pasteurization step, although omitting the pasteurization step also affords suitable products. If a pasteurization step is performed, it may be performed as step (i) or as step (c). In a preferred embodiment, a pasteurization step is performed, since this is a requirement for infant formula products in many jurisdictions from a food safety perspective. In a preferred embodiment, the process of the invention contains only a single pasteurization step to ensure the obtained product is sufficiently heat-treated with regards to prevention of microbial or bacterial contaminations but on the other hand ensures preservation of protein nativity. Thus, in a preferred embodiment, step (i) is a pasteurization step and step (c) is not performed, or step (i) is a filtration step and step (c) is performed. Although the incoming defatted milk may be pasteurized in step (i), it is preferred that if a pasteurization step is included, the recombined stream originating from step (b) is subjected to a pasteurization step (c) prior to being subjected to step (d). Alternatively, no pasteurization step is performed and step (i) is performed by filtration and step (c) in omitted. By virtue of filtration step (i), the thus obtained products are sufficiently debacterized to be suitable in the context of the present invention. Most preferably, step (c) is performed in case debacterialization in step (i) is achieved by microfiltration.

Pasteurization is known in the art and may e.g. involve HTST, ESL or UHT. The pasteurization step as meant herein has the purpose of reducing the microbial load to such an extent that the resulting infant formula product is free from microorganisms and safe for consumption by infants. In particular, it is safe with regards to Bacillus cereus and Enterobacter sakazakii, for instance, such as laid down in European Regulation No 2073/2005 dated 2007, corrigendum No. 1441/2007. Preferably, pasteurization involves heating at 72-74° C. for 15 to 30 seconds, or, alternatively, a heat-treatment equivalent thereto, meaning that the same heat load is applied, as is known to the skilled person. Preferably, the equivalent heat-treatment results in the same reduction in bacterial load and preserves the protein nativity to the same extent as a pasteurization step at 72-74° C. for 15 to 30 seconds, resulting in whey proteins having a nativity value of more than 90%, preferably more than 95 or even more than 98%.

### Step (d)

In step (d), the recombined stream originating from step (b) is used to manufacture the infant formula product. Such manufacturing is known in the art and typically involves one or more of drying, concentrating, supplementing with vitamins, minerals, lipids and/or dietary fibres, heat treatment, homogenisation, packaging. In a preferred embodiment, step (d) does not involve heat treatment, and involves one or more of drying, concentrating, supplementing with vitamins, minerals, lipids and/or dietary fibres and packaging. Preferably, step (d) involves at least a drying step, most preferably it involves all of the above mentioned steps. In a preferred embodiment, a drying step is performed directly after step (b) or (c), most preferably directly after step (c).

Although one or more of the separate streams may be dried prior to being combined in step (b), it is preferred that the recombined stream originating from step (b) is dried, preferably spray-dried. As such, only one drying step is needed in the manufacture of the infant formula product. In a preferred embodiment, the process according to the invention comprises only a single drying step, wherein in step (d) the recombined stream is dried, preferably by spray-drying. Due to the inherently limited heat-load as a consequence of low water activity of droplets produced during spray-drying, protein nativity remains substantially the same and is not significantly impacted during spray-drying. This allows that the content of native protein in the final infant formula product is as high as possible and substantially the same as prior to spray-drying. To retain the native protein content in the final product, the spray-drying step is preferably executed with an inlet temperature of less than 250° C., preferably less than 220° C., more preferably less than 200° C. Alternatively worded, the spray-drying step is executed such that the wet bulb temperature is kept below 80° C., preferably below 70° C. or even below 50° C. Using such spray-drying conditions, nativity of the proteins that are spray-dried will not be impacted anymore due to the low water activity of the infant formula powder particles in the spray-drier. In one embodiment, the recombined stream is concentrated, preferably prior to being dried. Such concentration may be accomplished by any means known in the art, such as by reverse osmosis (RO), nanofiltration (NF) and/or evaporation.

Depending on the desired type of infant formula product, supplementation of certain components, such as vitamins, minerals, lipids and/or dietary fibres, may be desired. Such supplementation can be performed either prior to, during or after combining step (b) and/or optionally prior to or after a drying step. The skilled person is aware of the requirements of particular types of infant formula products, e.g. from EU directive 91/321/EEC or EU directive 2006/141/EC or US Food and Drug Administration 21 CFR Ch 1 part 107, and is able to adjust the composition of the recombined stream in order to meet those requirements.

In one embodiment, the composition according to the invention is an infant formula product comprising whey protein, wherein the whey protein is intact and native. In one especially preferred embodiment, the composition according to this embodiment is obtainable by the process according to the invention as defined above.

As will be appreciated by the skilled person, process steps that lead to denaturation of the whey protein should be avoided as much as possible. For example, the infant formula may be a spray-dried powder, in which case it is preferred that that the spray-drying step is executed with an inlet temperature of less than 250° C., preferably less than 220° C., more preferably less than 200° C. It is preferred that the whey proteins have undergone a pasteurization step preferably a single pasteurization step.

The whey protein being “native”, is herein defined as having a nativity value of at least 90%, preferably at least 94%, most preferably at least 96%. In one embodiment, the nativity value is in the range of 90-100%, preferably in the range of 94-99%, more preferably in the range of 96-99%. In one embodiment, the nativity value is in the range of 90-99%, preferably in the range of 91-96%, more preferably in the range of 92-94%. The inventors found that both pasteurization and spray-drying not or only very slightly reduce the nativity value of the whey protein. As such, a whey protein is available that is save to be used in infant formulae but with limited allergenicity. Being the two most abundant whey proteins, it is especially preferred that α-lactalbumin and β-lactoglobulin have high nativity values. The inventors surprisingly found that especially β-lactoglobulin remained largely native in the process according to the present invention. It is thus preferred that α-lactalbumin has a nativity value of at least 70%, more preferably 75-95%, most preferably 78-85%. Likewise, it is preferred that β-lactoglobulin has a nativity value of at least 70%, more preferably 80-100%, most preferably 85-95%. Without being bound to a theory, it is believed that the nativity of α-lactalbumin and/or β-lactoglobulin, especially β-lactoglobulin, contribute to the beneficial effects on allergy.

Nativity values are known in the art and can be determined by any means available to the skilled person. The nativity value refers to the percentage of native protein of a particular type based on the total amount of protein of the same type. Here, the nativity value of the whey protein refers to the amount of native whey protein based on the total amount of whey protein. In one embodiment, the nativity value is determined according to the procedure in example 3.

In a second aspect, the invention concerns the infant formula product according to this embodiment, according to any of the definitions or preferred embodiments recited herein. This composition may be referred to as a hypoallergenic infant formula, since the severity of allergic response is significantly reduced. In one embodiment, the infant formula product shows a reduced allergic skin response, preferably a reduced food allergy response.

### Further Preferred Embodiments of the Infant Formula Product

The following applies to the composition according to the invention, irrespective whether it is defined by the process of manufacture or by the presence of whey protein which is intact and native. In an especially preferred embodiment, the composition according to the invention is defined by the process of preparing the composition and by the presence of an intact whey protein. In an especially preferred embodiment, the composition according to the invention is defined by the process of preparing the composition and by the presence of a native whey protein, as defined herein. In an especially preferred embodiment, the composition according to the invention is defined by the process of preparing the composition and by the presence of a native and intact whey protein, as defined herein.

Since the composition is an infant formula product, it is typically nutritionally complete for infants, and contains all necessary macronutrients and micronutrients for infant formula products as known in the art. Specifically, the infant formula product preferably contains casein, in addition to the native and intact whey protein. The whey protein to casein weight ratio in the infant formula product is preferably in the range of 90:10 to 40:60, more preferably in the range of 80:20 to 50:50, even more preferably in the range of 75:25 to 50:50, most preferably in the range of 70:30 to 55:45. In one embodiment, the whey protein to casein weight ratio in the in the infant formula product is about 60:40. The exact ratio is typically determined by the type of infant formula product that is being produced, and can be adjusted as known in the art. In a preferred embodiment, the whey protein, preferably all protein, has not been subjected to a hydrolysis step, wherein the protein is partly or fully hydrolysed. Likewise, it is preferred that the process for obtaining the infant formula product does not contain a hydrolysis step, wherein the whey protein, preferably all protein, is partly or fully hydrolysed.

In one embodiment, the composition according to the invention show a negative reaction to an alkaline phosphatase (ALP) activity test. Tests for alkaline phosphate activity are known in the art and are used as standard for defining the activity (or lack of activity) of the enzymes in an infant formula product. The law, for example European Regulation 2074/05, requires the ALP activity to be below 350 mU/L.

The ALP activity can be defined as mU/g (typically for powders, or for liquids based on dry weight) or mU/L (typically for liquids, including reconstituted powders). The ALP activity of the composition according to the invention, when in liquid form, is typically below 450 mU/L, preferably below 350 mU/L, or, when in powder form, is typically below 450 mU/L, preferably below 350 mU/L, after reconstitution as common in the art of infant formula products. In one embodiment, the composition according to the invention is liquid or reconstituted powder and has an ALP activity in the range of 0-450 mU/L, preferably 100-350 mU/L, more preferably 200-350 mU/L, most preferably 250-320 mU/L. Products having such ALP activities may be referred to as denatured and/or deactivated (see e.g. Example 1). Alternatively, the ALP may also be higher, such as more than 350, or in the range of 350-100000 mU/L, preferably 1000-50000 mU/L, most preferably 10000-30000 mU/L. In one embodiment, the ALP activity is in the range 350-450 mU/L. Products having such ALP activities may be referred to as native (see e.g. Example 1). In one embodiment, the composition according to the invention has an ALP activity of at most 20 mU/g, preferably at most 5 mU/g, or the composition according to the invention has an ALP activity in the range of 0-20 mU/g, preferably 0.1-10 mU/g, more preferably 0.2-7 mU/g, most preferably 0.5-5 mU/g, based on dry weight of the composition. In an alternative embodiment, the composition according to the invention has an ALP activity of at least 25 mU/g, preferably at least 30 mU/g, or the composition according to the invention has an ALP activity in the range of 25-150 mU/g, preferably 30-50 mU/g, based on dry weight of the composition.

In one embodiment, the ALP activity is determined by ISO standard 11816-1. Alternatively, the ALP activity is determined by the following procedure. A solution of the whey protein, typically as a 10 wt % protein solution, is mixed with an equal amount of 1-butanol, and the mixture is then centrifuged between 2500-3500 g for 30 min. The aqueous phase is collected from beneath the fat layer and diluted between 1/5-1/200. These sample solutions were added in the wells of an enzyme-linked immunosorbent assay (ELISA) plate, coated with a monoclonal antibody specific to the alkaline phosphatase found in cow's milk, together with control and standard solutions. The plates are incubated for 1 h at 18-25° C., after which the solutions are removed from the wells and substrate solution is added to each well. The plates are incubate for 2 h at 35-38° C. After stopping the incubation, the plate is imaged at a wavelength of 405 nm, and the ALP activity is determined by comparison of the optical density of the sample with that of the standard. In an especially preferred embodiment, the infant formula product according to the invention comprises intact whey protein, wherein at least 90% of the whey protein is native and the ALP activity is in the range of 100-350 mU/L, as determined by ISO standard 11816-1. Preferably, this infant formula product is obtainable by the process defined herein in case a pasteurization step is included to provide a debacterialized milk. In an alternative preferred embodiment, the infant formula product according to the invention comprises intact whey protein, wherein at least 90% of the whey protein is native and the ALP activity is higher than 350 mU/L or in the range of 350-100000 mU/L, as determined by ISO standard 11816-1. Preferably, this infant formula product is obtainable by the process defined herein in case a microfiltration step is included using a membrane capable of retaining bacteria and permeating milk proteins to provide a debacterialized milk.

### Application

The inventors surprisingly found that the composition according to the invention is capable of reducing and/or preventing allergic response. In a preferred embodiment, the allergic response is related to food allergy, in particular milk allergy, whey protein allergy or bovine milk allergy. The allergic response may be direct or indirect. Preferably, the allergic response is a direct or immediate response.

In an especially preferred embodiment, the allergic response is allergic skin response. In a preferred embodiment, the allergic skin response is related to food allergy, in particular milk allergy, whey protein allergy or bovine milk allergy. The allergic skin response may be direct or indirect. Preferably, the allergic skin response is a direct or immediate skin response.

Accordingly, in a first aspect, the invention concerns an infant formula product obtainable by a process comprising:
(a) processing defatted milk into a casein stream, a whey protein stream and a lactose stream, by:
   (i) subjecting the defatted milk to microfiltration over a membrane capable of retaining bacteria and permeating milk proteins or to a pasteurization step, to provide a debacterialized milk;
   (ii) subjecting the permeate originating from step (i) to microfiltration over a membrane capable of retaining casein and permeating whey proteins, to provide a casein stream as retentate and a permeate comprising whey protein;
   (iii) fractionating the permeate originating from step (ii) into a whey protein stream and a lactose stream;
(b) combining at least part of the casein stream, at least part of the whey protein stream originating from step (a) and a lactose source to obtain a recombined stream;
(c) optionally pasteurization of the recombined stream from step (b),
(d) using the recombined stream originating from step (b) or (c) in the manufacture of the infant formula product,
   for use in reducing and/or preventing allergic response.

The invention according to this aspect can also be worded as a use of defatted milk for the manufacture of an infant formula product for use in reducing and/or preventing allergic response, wherein the infant formula product is obtainable by a process comprising:
(a) processing defatted milk into a casein stream, a whey protein stream and a lactose stream, by:
   (i) subjecting the defatted milk to microfiltration over a membrane capable of retaining bacteria and permeating milk proteins or to a pasteurization step, to provide a debacterialized milk;
   (ii) subjecting the permeate originating from step (i) to microfiltration over a membrane capable of retaining casein and permeating whey proteins, to provide a casein stream as retentate and a permeate comprising whey protein;
   (iii) fractionating the permeate originating from step (ii) into a whey protein stream and a lactose stream;
(b) combining at least part of the casein stream, at least part of the whey protein stream originating from step (a) and a lactose source to obtain a recombined stream;
(c) optionally pasteurization of the recombined stream from step (b),
(d) using the recombined stream originating from step (b) or (c) in the manufacture of the infant formula product.

The invention according to this aspect can also be worded as a method for reducing and/or preventing allergic response, comprising administering to the subject an infant formula product obtainable by a process comprising:
(a) processing defatted milk into a casein stream, a whey protein stream and a lactose stream, by:
   (i) subjecting the defatted milk to microfiltration over a membrane capable of retaining bacteria and permeating milk proteins or to a pasteurization step, to provide a debacterialized milk;
   (ii) subjecting the permeate originating from step (i) to microfiltration over a membrane capable of retaining casein and permeating whey proteins, to provide a casein stream as retentate and a permeate comprising whey protein;
   (iii) fractionating the permeate originating from step (ii) into a whey protein stream and a lactose stream;
(b) combining at least part of the casein stream, at least part of the whey protein stream originating from step (a) and a lactose source to obtain a recombined stream;
(c) optionally pasteurization of the recombined stream from step (b),
(d) using the recombined stream originating from step (b) or (c) in the manufacture of the infant formula product.

Alternatively, the invention according to the first aspect concerns an infant formula product comprising whey protein, wherein the whey protein is intact and native, as defined by a nativity value of at least 90%, for use in reducing and/or preventing allergic skin response.

In other words, the invention according to the first aspect concerns the use of whey protein for the manufacture of an infant formula product for use in reducing and/or preventing allergic skin response, wherein the whey protein comprised in the infant formula product is intact and native, as defined by a nativity value of at least 90%.

In other words, the invention according to the first aspect concerns a method for reducing and/or preventing allergic response, comprising administering to the subject an infant formula product comprising whey protein, wherein the whey protein is intact and native, as defined by a nativity value of at least 90%.

In an alternative embodiment, the invention relates to an infant formula product, defined by the process of manufacture and/or by the presence of whey protein which is intact and native, for use in preventing or treating atopic dermatitis and/or eczema.

The invention according to this aspect is referred to as use according to the invention, which is equally applicable to composition for use according to the invention and the method according to the invention, as defined above.

The subject of the use according to the invention is typically an infant, preferably a human infant. Preferably, the infant is 0-36 months of age, more preferably 0-24 months of age, even more preferably 0-12 months of age, most preferably 0-6 months of age. In a preferred embodiment, the subject is in need of reducing and/or preventing allergic response. In one embodiment, the subject suffers from allergy. In one embodiment, the subject is at risk of developing allergy. Herein, the allergy is preferably food allergy, more preferably milk allergy and/or whey protein allergy, most preferably whey protein allergy. The milk allergy is typically bovine milk allergy, and the whey protein allergy is typically bovine whey protein allergy, especially in case defatted bovine milk is used as starting material of the process according to the invention. In one embodiment, the subject suffers from direct or immediate skin response or is at risk of developing direct or immediate skin response.

The infant formula product according to the invention is typically suitable as complete nutritional product for infants, like regular infant formula. Administration of the infant formula product according to the invention this occurs as (part of) the regular feeding regime of the infant. In one embodiment, the use according to the invention is further for providing nutrition to the infant.

### EXAMPLES

The following examples illustrate the invention.

### Example 1: WPC70 Preparation

Three WPC70 products, (i) native WPC70, (ii) deactivated WPC70, and (iii) denatured WPC70, were prepared according to the following process. Milk and subsequent fractions were stored at 4° C. throughout production. Whole raw milk (purchased from Dairygold) was skimmed using typical GEA Westfalia Separator @ 55° C. and cooled to 4° C. Skim milk was subjected to microfiltration to separate casein from both whey and lactose. Microfiltration membrane used was a 0.08 μM Synder membrane FR (PVDF 800 kDa) spiral wound membrane. The microfiltration retentate (MFR) was kept as the casein fraction and the microfiltration permeate (MFP) contained whey, lactose and ash. The operating temperature was 10° C. and volume concentration factor (VCF) was 3. This VCF factor was optimal to obtain the required final concentration of casein protein in the MFR. The MFP was then subjected to ultrafiltration to separate whey protein from lactose at operating temperature of 10° C. with VCF of 90. This VCF factor gave an optimal final concentration of whey protein in ultrafiltration retentate (UFR). A native WPC70 was produced. The ultrafiltration membrane used was a 10 kDa Synder membrane ST (PES 10 kDa) spiral wound membrane. Diafiltration medium was added to improve separation efficiency of membranes (200% of original starting skim milk volume). Concentrated liquid WPC70 (DM 11%) was stored at 4° C. until further handling. The WPC70 was heated to 30° C. and spray dried at 11% DM. The spray-dryer used was a single stage pilot scale dryer operated with an inlet temperature of 185° C. and outlet temperature of 90° C. This sample is referred to as the native WPC70 and represents a highly native, alkaline phosphatase positive sample.

Deactivated WPC70 was prepared to represent a highly native, pasteurized protein sample which can be included in an infant formula. It was prepared by re-hydrating the native WPC70 in 40° C. RO water using a high speed mixer for 30 min, resulting in a total solids content of 10% and a protein content of about 7%. This solution was heat-treated at 73° C./30 s using a Microthermics tubular heat exchanger (MicroThermics, North Carolina, USA). The heat-treated WPC was then freeze-dried resulting in a WPC70 powder with inactivated bioactive components, indicated by the inactivation of alkaline phosphatase, and whey protein nativity value of >95%.

Denatured WPC70 was prepared by re-hydrating the native WPC70 in 40° C. RO water using a high speed mixer for 30 min, resulting in a total solids content of 10% and a protein content of about 7%. This solution was heat treated at 100° C./60 s using a Microthermics tubular heat exchanger (MicroThermics, North Carolina, USA). The heat-treated WPC was then freeze-dried resulting in a WPC70 powder with whey protein nativity value of <30%.

The composition of the three WPC70 products, as a 7% protein solution (see Example 3), is given in the table below (in wt % based on dry weight):

| | Total | | | True | | | |
|---|---|---|---|---|---|---|---|
| | Protein | NPN | NCN | protein | Casein | Whey | Nativity |
| Native | 7.13 | 0.15 | 5.33 | 6.97 | 1.65 | 5.30 | 100% |
| WPC70 | | | | | | | |
| Deactivated | 7.11 | 0.15 | 5.23 | 6.96 | 1.73 | 5.08 | 95.73% |
| WPC70 | | | | | | | |
| Denatured | 6.70 | 0.16 | 1.16 | 6.54 | 5.38 | 1.14 | 21.43% |
| WPC70 | | | | | | | |

### Example 2: IMF Preparation

Three IMF products, (i) Native IMF, (ii) Deactivated IMF, and (iii) denatured IMF, were prepared according to the following process. The wet phase of the infant milk formulation was prepared by first dissolving lactose powder in 90° C. RO water with agitation provided by a high speed silverson mixer (Silverson®, Chesham Bucks, U.K). The solution was cooled to 45° C., micellar casein concentrate (MCC, MFR obtained in Example 1) and native whey protein concentrate (native WPC70 obtained in Example 1) were added to the solution, allowing for a final casein:whey ratio of 40:60 (similar to the ratios observed in mothers milk), and re-hydrated under high speed mixing for 20 min. Galacto-oligossaccharide (GOS) syrup was added to the mix once the casein and whey protein powders had sufficiently hydrated and mixed for 15 min. Micronutrient components were added to the macronutrients as per a pre-determined recipe. All ingredients were added and agitated at high speeds for 20 min.

For native IMF, the wet phase was directly combined with a pre-prepared oil blend and homogenized via the addition of soy lecithin powder and high speed agitation for 20 min. This completed IMF (50-55% TS) was subjected directly to a multi-stage Anhydro spray-dryer (water evaporation capacity (WEC) 30 kg/hr) operated with an inlet temperature of 185° C. and an outlet temperature of 90° C., resulting in a powdered native IMF with <4% moisture.

Deactivated IMF was manufactured by pasteurizing the wet phase using a Microthermics tubular heat exchanger (MicroThermics, North Carolina, USA) at 73° C./30 s. The pasteurized wet phase was combined with a pre-prepared oil blend and homogenized via the addition of soy lecithin powder and high speed agitation for 20 min. This pasteurized compound was dried using a Single stage pilot dryer (WEC 10 kg/hr) which produced a deactivated IMF with a whey protein nativity value of >95% and inactivated bioactive components as indicated by the inactivation of the enzyme alkaline phosphatase.

Denatured IMF was prepared by rehydration of native IMF powder to a protein content of about 10%. This compound was mixed at high speed for 30 min to ensure full dissolution. The compound was then heat-treated using a Microthermics tubular heat exchanger (MicroThermics, North Carolina, USA) at 100° C./60 s. The heat-treated compound was collected and freeze-dried to produce a denatured IMF with a whey protein nativity value of <40%.

The composition of the three IMF products, as a 10% protein solution (see Example 3), is given in the table below (in wt % based on dry weight):

| | Total | | | True | | | |
|---|---|---|---|---|---|---|---|
| | Protein | NPN | NCN | protein | Casein | Whey | Nativity |
| Native | 10.19 | 0.41 | 6.44 | 9.78 | 3.34 | 6.38 | 100% |
| IMF | | | | | | | |
| Deactivated | 10.05 | 0.40 | 6.25 | 9.65 | 3.40 | 6.19 | 97.03% |
| IMF | | | | | | | |
| Denatured | 9.98 | 0.40 | 2.43 | 9.59 | 7.15 | 2.37 | 37.16% |
| IMF | | | | | | | |

### Example 3: Nativity Value Calculation

The total nitrogen (TN), non-protein nitrogen (NPN) and non-casein nitrogen (NCN) were determined via kjeldahl analysis, as per the ISO 8968-3/IDF 20-3:2004 standard (Milk—Determination of nitrogen content—Part 3: Block-digestion method (Semi-micro rapid routine method), 2004), using an automatic Kjeltec 8400 unit (FOSS, Warrington, U.K). The nativity values of the whey proteins in Examples 1 and 2 were calculated as follows:

Casein fraction=(TP−NPN)−NCN (a)

Whey fraction=NCN−NPN (b)

Nativity value=measured whey fraction (b)/theoretical whey faction*100% (c)
The theoretical whey fraction is based on the casein/whey protein ratio of the product, from the recipe of the product.

### Example 4: Allergenicity Native and Denatured WPC70 Products Obtained in Example 1

Four-week-old, specific pathogen free, female C3H/HeOuJ mice were purchased at Charles River Laboratories (The Netherlands) and housed at the animal facility of the Utrecht University on a 12 h light/dark cycle with access to food and water ad libitum. All animal procedures were conducted according to governmental guidelines and approved by the Ethical Committee for Animal Research of the Utrecht University, Utrecht, The Netherlands (CCD: AVD108002015346).

After one week habituation, mice (n=8/group) were sensitized intragastrically (i.g.) using a blunt needle with 20 mg native (non-heated) or denatured (heated) WPC70 obtained according to Example 1, in 0.5 mL PBS containing 10 μg cholera toxin (CT; List Biological Laboratories, Campbell, USA) as an adjuvant. Sham-sensitized control mice (n=6) received CT alone (10 μg/0.5 mL PBS). Mice were sensitized once a week for 5 consecutive weeks (on day 0, 7, 14, 21 and 28) as previously described by van Esch et al. (Pediatr Allergy Immunol (2011) 22(8):820-6). Five days after the last sensitization (day 33), mice were challenged intradermally (i.d.) in the ear pinnae of both ears with 10 μg denatured WPC70 of Example 1 in 20 μl PBS to determine the acute allergic skin response by locally measuring swelling of the skin of the ears via the thickness of the skin. On the same day, mice were challenged i.g. with 50 mg denatured WPC70 in 0.5 mL PBS. 18 h after the oral challenge blood samples were collected and centrifuged at 10.000 rpm for 10 min. Serum was obtained and stored at −20° C. until further analysis. Mice were killed by cervical dislocation and samples were obtained for ex vivo analysis.

Blood was collected via cheek puncture 18 h after oral challenge with denatured WPC70 and centrifuged at 10.000 rpm for 10 min. Serum was obtained and stored at −20° C. until analysis of whey-specific IgE levels by means of ELISA. Determination of whey-specific IgE antibodies was performed as previously described (Schouten et al. Int Arch Allergy Immunol (2008) 147(2): 125-34), with few alterations. Briefly, high binding Costar 9018 plates (Corning Inc., New York, USA) were coated with 20 μg/mL whey protein in carbonate/bicarbonate coating buffer (0.05 M, pH 9.6; Sigma-Aldrich, Zwijndrecht, The Netherlands) and incubated overnight at 4° C. After overnight incubation, plates were washed and blocked for 1 h with PBS/1% bovine serum albumin (BSA; Sigma-Aldrich). Serum samples were subsequently incubated for 2 h. After washing, plates were incubated with biotinylated rat anti-mouse IgE detection antibody (1 μg/mL; BD Biosciences, Alphen aan de Rijn, The Netherlands) for 1.5 h. Plates were then washed, incubated for 45 min with streptavidin-horse radish peroxidase (0.5 μg/mL; Sanquin, Amsterdam, The Netherlands), washed again and developed using o-phenylendiamine (Sigma-Aldrich). The reaction was stopped with 4 M H₂SO₄ and absorbance was measured at 490 nm on a microplate reader (Bio-Rad, Veenendaal, The Netherlands).

Single cell splenocyte suspensions were obtained by passing spleen samples through a 70-μm nylon cell strainer using a syringe. The splenocyte suspension was rinsed with RPMI 1640 medium (Lonza, Verviers, Belgium) and incubated with lysis buffer (8.3 g NH₄Cl, 1 g KHC₃O and 37.2 mg EDTA dissolved in 1 L demi water, filter sterilized) to remove red blood cells. The reaction was stopped by adding RPMI 1640 medium supplemented with 10% heat-inactivated fetal bovine serum (FBS; Bodinco, Alkmaar, The Netherlands), penicillin (100 U/mL)/streptomycin (10 mg/mL; Sigma-Aldrich) and β-marcaptoethanol (20 μM; Thermo Fisher Scientific, Paisley, Scotland). Splenocytes were subsequently resuspended in this culture medium. For the ex vivo antigen-specific restimulation assay, splenocytes (8×10⁵ cells/well) were cultured in culture medium with or without 500 μg/mL whey. Supernatant was harvested after 4 days of culture (37° C., 5% CO₂) and stored at −20° C. until cytokine analysis. Measurements of IL-5 and IL-13 were performed by means of ELISA according to the protocol described above for IgE. Purified rat anti-mouse antibodies (1 μg/mL for IL-5 and 2 μg/mL for IL-13), recombinant mouse cytokines and biotinylated rat anti-mouse antibodies (1 μg/mL for IL-5 and 400 ng/mL for IL-13) were purchased at BD Biosciences.

Statistical analysis: Data below are presented as mean±SEM. Differences compared to the hWP-hWP group were statistically analyzed using one-way ANOVA, followed by Dunnett's multiple comparisons test. Serum IgE levels were analyzed using Kruskal-Wallis test for non-parametric data followed by Dunn's multiple comparisons test since data did not obtain normality. Results were considered statistically significant when p<0.05. Analyses were performed using GraphPad Prism software (version 7).

The results on IgE levels generated in the present model are indicative of a lower systemic sensitivity to the allergen used. Low levels of IL5 and IL13 (two Th2 cytokines) are indicative of reduced allergenicity of the tested WPC70 and thus also an infant formula containing these isolated whey proteins. Results similar to the denatured WPC70 were obtained when a standard infant formula purchased from the supermarket was used. In particular, significantly higher ear swelling was measured, as well as high IgE levels and increased mast cell degranulation for the presently marketed infant formula. The results are depicted in the table below:

| | Acute allergic | Mast cell | WPC70- | | |
|---|---|---|---|---|---|
| | skin response | degranulation | specific IgE | IL-5 | IL-13 |
| | (μm) | (ng/mL) | (OD) | (pg/mL) | (pg/mL) |
| PBS | 43.46 ± 7.13 **** | 26.12 ± 3.68 ** | 0.002 ± 0.002 *** | 0.00 ± 0.00 ** | 4.66 ± 4.66 ** |
| Denatured | 166.90 ± 7.14 | 173.40 ± 36.94 | 0.709 ± 0.169 | 309.60 ± 96.23 | 629.30 ± 185.70 |
| WPC70 | | | | | |
| Native | 106.30 ± 9.76 *** | 60.54 ± 13.24 ** | 0.371 ± 0.229 | 67.26 ± 27.40 * | 126.70 ± 34.75 ** |
| WPC70 | | | | | |

Data are presented as mean ± SEM, n = 6 in PBS group and n = 7-8 in all other groups.
* P < 0.05,
** P < 0.01,
*** P < 0.001,
**** P < 0.0001 compared to the denatured WPC70 group as analyzed with one-way ANOVA followed by Dunnett's multiple comparisons test or Kruskal-Wallis test for non-parametric data followed by Dunn's multiple comparisons test.

Results similar to the above-mentioned cross-over set-up where obtained when the challenge was performed with the same protein sample with which the sensitization was done.

### Example 5: Allergenicity IMF Products Obtained in Example 2

Four-week-old, specific pathogen free, female C3H/HeOuJ mice were purchased at Charles River Laboratories (The Netherlands) and housed at the animal facility of the Utrecht University on a 12 h light/dark cycle with access to food and water ad libitum. All animal procedures were conducted according to governmental guidelines and approved by the Ethical Committee for Animal Research of the Utrecht University, Utrecht, The Netherlands (CCD: AVD108002015346).

After one week habituation, mice (n=8/group) were sensitized intragastrically (i.g.) using a blunt needle with the native, deactivated or denatured infant milk formula (IMF) of Example 2, containing 20 mg protein (1856 mg native IMF, 1714 mg deactivated IMF) in 0.5 mL PBS containing 10 μg cholera toxin (CT; List Biological Laboratories, Campbell, USA) as an adjuvant. Sham-sensitized control mice (n=6) received CT alone (10 μg/0.5 mL PBS). Mice were sensitized once a week for 5 consecutive weeks (on day 0, 7, 14, 21 and 28) as previously described by van Esch et al. (Pediatr Allergy Immunol (2011) 22(8):820-6). Five days after the last sensitization (day 33), mice were challenged intradermally (i.d.) in the ear pinnae of both ears with 10 μg denatured WPC70 in 20 μl PBS to determine the acute allergic skin response by locally measuring swelling of the skin of the ears via the thickness of the skin. On the same day, mice were challenged i.g. with 50 mg denatured WPC70 in 0.5 mL PBS. 18 h after the oral challenge blood samples were collected and centrifuged at 10.000 rpm for 10 min. Serum was obtained and stored at −20° C. until further analysis. Mice were killed by cervical dislocation and samples were obtained for ex vivo analysis.

Blood was collected via cheek puncture 18 h after oral challenge and centrifuged at 10.000 rpm for 10 min. Serum was obtained and stored at −20° C. until analysis of whey-specific IgE and mouse mast cell protease-1 (mMCP-1) levels by means of ELISA. Determination of whey-specific IgE antibodies was performed as previously described (Schouten et al. Int Arch Allergy Immunol (2008) 147(2): 125-34), with few alterations. Briefly, high binding Costar 9018 plates (Corning Inc., New York, USA) were coated with 20 μg/mL WPC70 in carbonate/bicarbonate coating buffer (0.05 M, pH 9.6; Sigma-Aldrich, Zwijndrecht, The Netherlands) and incubated overnight at 4° C. After overnight incubation, plates were washed and blocked for 1 h with PBS/1′Y° bovine serum albumin (BSA; Sigma-Aldrich). Serum samples were subsequently incubated for 2 h. After washing, plates were incubated with biotinylated rat anti-mouse IgE detection antibody (1 μg/mL; BD Biosciences, Alphen aan de Rijn, The Netherlands) for 1.5 h. Plates were then washed, incubated for 45 min with streptavidin-horse radish peroxidase (0.5 μg/mL; Sanquin, Amsterdam, The Netherlands), washed again and developed using o-phenylendiamine (Sigma-Aldrich). The reaction was stopped with 4 M H₂SO₄ and absorbance was measured at 490 nm on a microplate reader (Bio-Rad, Veenendaal, The Netherlands). Concentrations of mMCP-1 were measured using a mMCP-1 Ready-SET-Go! ELISA (eBioscience, Breda, The Netherlands) according to the manufacturer's instructions.

Statistical analysis: Data are presented as mean±SEM. IMFs were compared to the native IMF group and differences were statistically analyzed using one-way ANOVA, followed by Dunnett's multiple comparisons test. Serum IgE levels were analyzed using Kruskal-Wallis test for non-parametric data followed by Dunn's multiple comparisons test since data did not obtain normality. Results were considered statistically significant when p<0.05. Analyses were performed using GraphPad Prism software (version 7).

Results indicate that the native and deactivated (i.e. pasteurized) IMF samples elicit lower allergenic responses to antigen exposure in an in vivo setting. The results are depicted in the table below:

| | Acute allergic skin response | WPC70-specific IgE |
|---|---|---|
| | (μm) | (OD) |
| PBS | 39.04 ± 7.50 | 0.001 ± 0.001 |
| Native IMF | 99.25 ± 12.98 | 0.027 ± 0.013 |
| Deactivated IMF | 58.68 ± 6.78 | 0.134 ± 0.062 |
| Denatured IMF | 150.4 ± 17.82 * | 0.301 ± 0.090 * |

Data are presented as mean ± SEM, n = 6 in PBS group and n = 8 in all other groups.
* P < 0.05,
**P < 0.01 compared to the native IMF group as analysed with one-way ANOVA followed by Dunnett's multiple comparisons test or Kruskal-Wallis test for non-parametric data followed by Dunn's multiple comparisons test.

Similar results to the denatured sample were obtained when a currently marketed standard infant formula was tested. Results similar to the above-mentioned cross-over set-up where obtained when the challenge was performed with the same protein sample with which the sensitization was performed.

### Example 6: Determination of Alkaline Phosphatase Activity

Alkaline phosphatase (ALP) activity in native and heat treated WPC and IMF products was determined using an immunocapture assay using specialized ALP assay kits (IDBiotech, Rue Marie Curie, Issoire, France). The kit contained an enzyme-linked immunosorbent assay (ELISA) plate coated with a monoclonal antibody specific to the alkaline phosphatase found in cow's milk. 1-butanol is the solvent used for enzyme extraction. Enzyme activity is expressed as equivalent-milliunit per litre (Eq·mU/l).

Sample preparation: 3 ml of WPC (10% protein) or IMF (10% protein) was mixed with 3 ml of 1-butanol, capped and mixed using a vortex for 30-40 s. Samples were then centrifuged between 2500-3500 g for 30 min. The aqueous phase was collected from beneath the fat layer and diluted between 1/5-1/200 (recommended) using the dilution buffer provided.

A standard solution was prepared as per the instructions within the assay kit, resulting in a working solution of 15,000 Eq·mU/l which in turn was diluted using the provided dilution buffer to create standards varying in concentration from 5,000-100 Eq·mU/l:
STD15000 Eq·mU/l: 125 μl (15,000 Eq·mU/l)+250 μl dilution buffer;
STD23000 Eq·mU/l: 75 μl (15,000 Eq·mU/l)+300 μl dilution buffer;
STD31000 Eq·mU/l: 25 μl (15,000 Eq·mU/l)+350 μl dilution buffer;
STD4500 Eq·mU/l: 50 μl (STD1)+450 μl dilution buffer;
STDS 300 Eq·mU/l: 50 μl (STD2)+450 μl dilution buffer;
STD6100 Eq·mU/l: 50 μl (STD3)+450 μl dilution buffer.

To run the assay: each well of the ELISA strips was washed with 300 μl of wash buffer, which was removed by inverting the plate. This is repeated 4 times. 100 μl of standard, control and sample solutions were added to the corresponding wells, the plate was covered and was shaken gently for 1 min and incubated for 1 hour at 18-25° C. After incubation, the standard, control and sample solutions were removed from the wells (by inversion of the plate) and the wash step as described above was performed. 100 μl of substrate solution was added to each well. The plate was covered, shaken gently for 1 min and incubate for 2 hrs at 35-38° C. A yellow colour develops. 50 μl of stop solution (provided in the kit) was added to all wells after incubation. The plastic cover was removed and the plate was read at 405 nm using a microplate reader. A calibration curve was obtained by plotting the optical density reading for the standard samples and this curve was used to determine the alkaline phosphatase activity in the WPC and IMF products.

The results are given in the table below:

| | ALP activity | |
|---|---|---|
| Native WPC70 | 195 | mU/g |
| Deactivated WPC70 | Not determined | |
| Native IMF | 33 | mU/g |
| Deactivated IMF | <3 | mU/g |

### Example 7: Determination of Alkaline Phosphatase Activity

The alkaline phosphatase (ALP) activity in native WPC and IMF products according to the invention (without and with denaturation) was determined in mU/L via ISO standard 11816-1 (version valid in October 2018). Solutions were prepared and tested according to the test protocol. The results for all four products at 1.3 wt % protein (based on total weight), which is the protein content of standard infant formulae, are given in the table below:

| | ALP activity | |
|---|---|---|
| Native WPC70 | 1.8 × 10⁴ | mU/L |
| Denatured WPC70 | <20 | mU/L |
| Native IMF | 2.1 × 10⁴ | mU/L |
| Denatured IMF | <20 | mU/L |

### Example 8: Determination of the Nativity Value of Commercial Infant Formula

Commercially available infant formulae were purchased from local stores in the fall of 2018 and tested for nativity well before the expiration date on the package. All formulae contained intact protein only, and no partial of fully hydrolysed protein. The nativity value of the whey proteins comprised in these formula was determined according to the procedure of example 3. The results are presented in the table below.

| | | | | | Nativity |
|---|---|---|---|---|---|
| | C:W * | TP | NPN | NCN × 6.25 | (%) |
| Frisolac Prestige 1 | 40:60 | 10.8 | 0.21 | 4.38 | 64.4 |
| Hipp Combiotik Bio 1 | 45:55 | 9.69 | 0.20 | 4.19 | 74.8 |
| Nestle Nan Optipro 1 | 36:64 | 9.61 | 0.20 | 2.81 | 42.5 |
| Hero Nutrasense 1 | 50:50 | 9.95 | 0.20 | 1.94 | 34.9 |
| Nestle Beba Optipro 1 | 38:62 | 10.02 | 0.20 | 2.44 | 36.0 |
| AusNutria Neolac Bio 1 | 47:53 | 11.5 | 0.20 | 3.81 | 59.3 |

* casein to whey protein weight ratio derived from the label of the product

1-21. (canceled)

22. A method for reducing and/or preventing allergic response, comprising administering to a subject in need thereof an infant formula product comprising intact whey protein for use in reducing and/or preventing allergic response, wherein at least 90% of the whey protein is native.

23. The method according to claim 22, wherein the allergic response is allergic skin response.

24. The method according to claim 23, wherein the skin response is allergic skin response related to food allergy, in particular milk allergy, whey protein allergy or bovine milk allergy.

25. The method according to claim 22, wherein the allergic response is an direct or immediate response.

26. The method according to claim 22, wherein the infant formula product is:
(a) a powder intended to be reconstituted into a liquid infant formula; or
(b) a liquid infant formula.

27. The method according to claim 22, wherein the infant formula product is obtained from defatted milk or debacterialized defatted milk.

28. The method according to claim 22, wherein the infant formula product is not subjected to heat treatment and/or wherein the infant formula product exhibits an alkaline phosphatase activity of at least 25 mU/g.

29. The method according to claim 22, wherein the infant formula product is a pasteurized infant formula product and/or wherein the infant formula product exhibits an alkaline phosphatase activity of at most 20 mU/g.

30. The method according to claim 22, wherein the infant formula product is a pasteurized infant formula product and/or wherein the infant formula shows a negative reaction to an alkaline phosphatase test.

31. The method according to claim 22, wherein the whey protein is obtained by subjecting defatted milk to consecutive filtration steps to obtain a whey protein stream.

32. The method according to claim 31, wherein the whey protein is obtained by:
(i) subjecting the defatted milk to microfiltration over a membrane capable of retaining bacteria and permeating milk proteins or to a pasteurization step, to provide a debacterialized milk;
(ii) subjecting the permeate originating from step (i) to microfiltration over a membrane capable of retaining casein and permeating whey proteins, to provide a casein stream as retentate and a permeate comprising whey protein; and
(iii) fractionating the permeate originating from step (ii) into a whey protein stream and a lactose stream.

33. The method according to claim 22, wherein at least 94% of the whey protein is native.

34. The method according to claim 22, wherein at least 70% of the α-lactalbumin is native and/or at least 70% of the β-lactoglobulin is native.

35. A method for reducing and/or preventing allergic response, comprising administering to a subject in need thereof an infant formula product comprising intact whey protein, wherein the infant formula product is obtainable by a process comprising:
(a) processing defatted milk into a casein stream, a whey protein stream and a lactose stream, by:
(i) subjecting the defatted milk to microfiltration over a membrane capable of retaining bacteria and permeating milk proteins or to a pasteurization step, to provide a debacterialized milk;
(ii) subjecting the permeate originating from step (i) to microfiltration over a membrane capable of retaining casein and permeating whey proteins, to provide a casein stream as retentate and a permeate comprising whey protein;
(iii) fractionating the permeate originating from step (ii) into a whey protein stream and a lactose stream;
(b) combining at least part of the casein stream, at least part of the whey protein stream originating from step (a) and a lactose source to obtain a recombined stream;
(c) optionally pasteurizing the recombined stream from step (b),
(d) using the recombined stream originating from step (b) or (c) in the manufacture of the infant formula product.

36. The method according to claim 35, wherein the infant formula product is a powder intended to be reconstituted into a liquid infant formula, the powder is a spray-dried powder, and wherein the spray-drying is part of step (d).

37. The method according to claim 35, wherein at least part of the lactose stream originating from step (a) is used as lactose source in step (b).

38. The method according to claim 35, wherein step (iii) is performed by ultrafiltration over a membrane capable of retaining whey proteins and permeating lactose, to provide a whey protein stream as retentate and a permeate comprising lactose.

39. The method according to claim 35, wherein step (iii) is performed by ultrafiltration operating with a volume concentration factor in the range of 20-200.

40. The method according to claim 35, wherein the defatted milk is the sole protein source for the infant formula product.

41. The method according to claim 35, wherein the manufacturing of step (d) includes at least one of drying, concentrating, supplementing with vitamins, minerals, lipids and/or dietary fibres, packaging.
